(19) United States
(12) Patent Application Publication
(10) Pub. No.: US 2016/0325102 A1
(43) Pub. Date: Nov. 10, 2016

(54) CARDIAC REGULATING APPARATUS AND REGULATING METHOD THEREOF

(71) Applicant: NATIONAL CHIAO TUNG UNIVERSITY, Hsinchu City (TW)

(72) Inventors: Chia-Chi CHANG, Hsinchu City (TW); Tzu-Chien HSIAO, Hsinchu City (TW); Hung-Yi HSU, Taichung City (TW)

(21) Appl. No.: 14/751,156

(22) Filed: Jun. 26, 2015

(30) Foreign Application Priority Data
May 4, 2015 (TW) 104114161

(51) Int. Cl. A61N 1/365; A61N 1/37

(52) U.S. Cl. CPC A61N 1/36564; A61N 1/3702

(57) ABSTRACT

A cardiac regulating apparatus and a cardiac regulating method are disclosed. The cardiac regulating apparatus includes a control unit, a sensing unit and an analyzing unit. The control unit is configured for outputting a cardiac control signal in accordance with a heart rate of a heart. The sensing unit is configured for acquiring a blood pulse signal. In response to the cardiac control signal having a first constant cardiac rhythm, the analyzing unit is configured for analyzing amplitude and reflected wave time of the blood pulse signal, and for outputting a regulation parameter according to the amplitude and the reflected wave time of the blood pulse signal. The control unit is configured for regulating the cardiac control signal according to the regulation parameter from the first constant cardiac rhythm to a second constant cardiac rhythm different from the first constant cardiac rhythm.

### RELATED APPLICATIONS

This application claims priority to Taiwanese Application Serial Number 104114161, filed May 4, 2015, which is herein incorporated by reference.

### BACKGROUND

1. Field of Invention

The present disclosure relates to a cardiac regulating apparatus and a cardiac regulating method thereof. More particularly, the present disclosure relates to a cardiac regulating apparatus, which regulates according to a blood pulse signal, and a cardiac regulating method thereof.

2. Description of Related Art

A typical cardiac pacemakers are designed as open-loop systems, which defaults with no functions of sensing and feedback controlling. Generally, the evaluation to a patient for a period of time, the cardiac pacemaker is implanted into the body of a patient with settings of the most suitable cardiac rhythm. After the implantation, the cardiac pacemaker is going to keep utilizing the fixed cardiac rhythm to stimulate the heart muscle.

However, the fixed cardiac rhythm may induce many physiological effects for the patient. For example, while the blood pressure is too low, the heart beat cannot increase to recover the blood pressure for the cardiac rhythm is fixed, which results in temporary oxygen deficiency for the patient. Similarly, while the patient is doing exercise, the heart beat is not allowed to regulate itself to improve metabolism and alleviate the stress due to the exercise.

The abovementioned physiological effects may further cause damage to the philological tissues. For example, long-term oxygen deficiency may influence intelligence and consciousness, and the constantly out-of-control blood pressure may cause cardiovascular diseases, such as damages to vascular walls or heart valves, oxygen deficiency of heart muscle and imbalance of blood pressure.

Currently adopted methods for evaluating autonomic nervous systems is either to dispose sensing circuits directly on the autonomic nervous systems, or to evaluate regulation conditions through parameters related to heart rate variability (HRV). However, the pacemaker-implanted patients would not have the HRV for the evaluation because of the fixed cardiac rhythm of the pacemaker. Therefore, the most difficult thing at the present time is to acquire the regulation parameters from the fixed cardiac rhythm patients with the cardiac pacemaker.

### SUMMARY

The following presents a simplified summary of the disclosure in order to provide a basic understanding to the reader. This summary is not an extensive overview of the disclosure and it does not identify key/critical components of the present disclosure or delineate the scope of the present disclosure. Its sole purpose is to present some concepts disclosed herein in a simplified form as a prelude to the more detailed description that is presented later.

One aspect of the present disclosure is to provide a cardiac regulating apparatus. The cardiac regulating apparatus includes a control unit, a sensing unit and an analyzing unit. The control unit is configured for outputting a cardiac control signal in accordance with a heart rate of a heart. The sensing unit is configured for acquiring a blood pulse signal. The analyzing unit, in response to the cardiac control signal having a first constant cardiac rhythm, is configured for analyzing amplitude and reflected wave time of the blood pulse signal, and outputting a regulation parameter according to the amplitude and the reflected wave time of the blood pulse signal. The control unit is configured for regulating the cardiac control signal according to the regulation parameter from the first constant cardiac rhythm to a second constant cardiac rhythm different from the first constant cardiac rhythm.

Another aspect of the present disclosure is to provide a cardiac regulating method. The method includes: outputting a cardiac control signal in accordance with a heart rate of a heart; acquiring a blood pulse signal; analyzing amplitude and reflected wave time of the blood pulse signal in response to the cardiac control signal having a first constant cardiac rhythm; outputting a regulation parameter according to the amplitude and the reflected wave time of the blood pulse signal; and regulating the cardiac control signal according to the regulation parameter from the first constant cardiac rhythm to a second constant cardiac rhythm different from the first constant cardiac rhythm.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a schematic diagram illustrating a blood pulse signal in a human body;

FIG. 2 is a schematic diagram illustrating amplitude and reflected wave time of a blood pulse signal according to an embodiment of the present disclosure;

FIG. 3 is a schematic diagram illustrating a cardiac regulating apparatus according to an embodiment of the present disclosure;

FIG. 4 is a schematic diagram illustrating a spectrum value according to an embodiment of the present disclosure; and

FIG. 5 is a schematic diagram illustrating a cardiac regulating method according to an embodiment of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

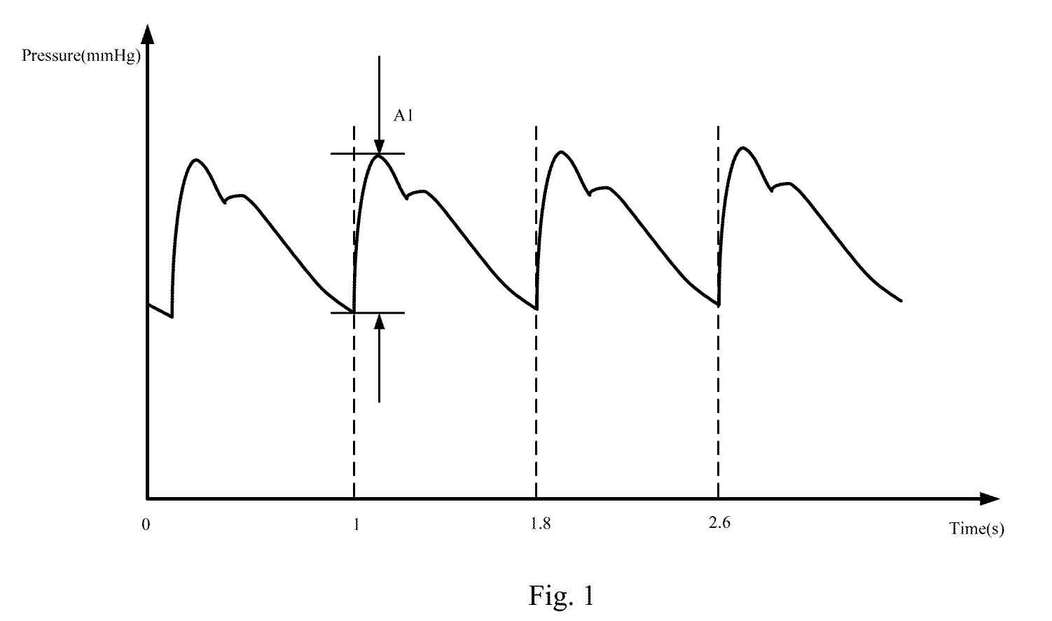

FIG. 1 is a schematic diagram illustrating a blood pulse signal in a human body. Generally, a heart rate in a human body has a constant cardiac rhythm such as 70 Hz˜80 Hz, and a blood pulse signal can be acquired from blood pressure, blood volume or pulse diagnoses. The blood pulse signal shown in FIG. 1 is an arterial blood pressure. It can be seen that the arterial blood pressure may periodically change with the constant cardiac rhythm, in which the pressure difference between the highest and the lowest blood pressure in one period is amplitude A1. However, the cardiac rhythm is not always kept constant because the heart can regulate the cardiac rhythm according to the physiological needs.

For example, during sports, oxygen in the human body is consumed rapidly, and thus muscles of bronchi will be relaxed and the cardiac rhythm will be increased by stimulating autonomic nervous systems (i.e., the sympathetic nervous system and the parasympathetic nervous system, and here means the sympathetic nervous system) for rapidly transmitting the oxygen to every place in the human body. For another example, while an ejection fraction in the human body is too low, that is to say, while the quantity of blood sent out from the heart is too low, the cardiac rhythm will also be increased by stimulating the autonomic nervous systems.

Therefore, if the information indicating that whether the autonomic nervous systems are stimulated or not can be obtained from bodies of patients implanted with pacemakers, a cardiac control signal can be regulated according to the information. For example, the cardiac rhythm may be increased while the sympathetic nervous system is stimulated, and the cardiac rhythm may be decreased while the parasympathetic nervous system is stimulated. It should be noted that, the current method utilized to measure that whether the autonomic nervous systems are stimulated or not is to dispose sensing circuits on the autonomic nervous systems, but the method can be carried out only by intrusive therapies.

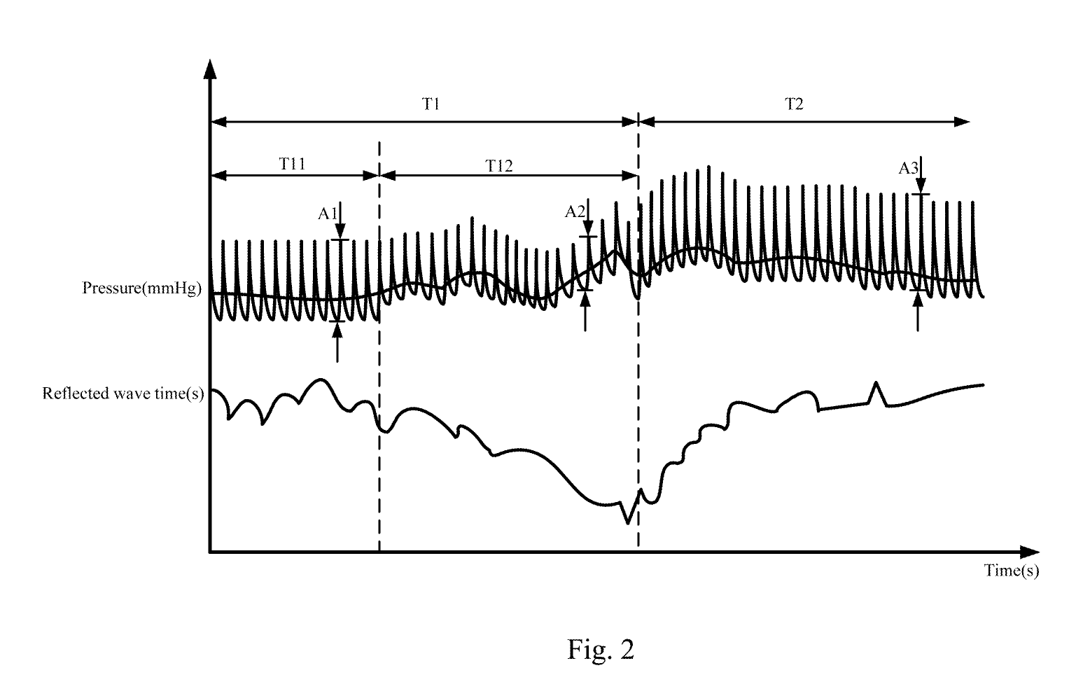

Thus, the present disclosure provides a non-intrusive method to measure the sympathetic nervous system and the parasympathetic nervous system. FIG. 2 is a schematic diagram illustrating amplitude and reflected wave time of a blood pulse signal according to an embodiment of the present disclosure. In this embodiment, in period T1, the human body is performing a valsalva maneuver, which means exerting and stopping breathing continuously, and this can be seen in everyday life, such as cough, vomit, defecation or raising weights, but the present disclosure is not limited in this regard.

As shown in FIG. 2, the value of the arterial blood pressure is kept rippling steadily and periodically in the initial period T11 i.e., the amplitude of the arterial blood pressure is kept as the amplitude A1. It should be noted that, while the blood pulse signal is transmitted in blood vessels, every pulse of the blood pulse signal can be regarded as including two portions. One of the portions is a traveling wave (which is not illustrated in FIG. 2) transmitted from the heart to end terminals of the blood vessels (e.g., wrists, necks, ankles, etc.) The other one of the portions is a reflected wave (which is not illustrated in FIG. 2) reflected by the end terminals of the blood vessels and transmitted to the heart. Therefore, reflected wave time shown in FIG. 2 is a curve illustrating time variations of the reflected wave.

In addition, the amplitude of the blood pulse signal can represent the magnitude of the ejection fraction, for example, while the amplitude of the blood pulse signal decreases, the ejection fraction also decreases, i.e., the quantity of blood sent out from the heart decreases. On the other hand, while the amplitude of the blood pulse signal increases, the ejection fraction also increases, i.e., the quantity of blood sent out from the heart increases. Besides, the reflected wave time of the blood pulse signal can represent that whether the sympathetic nervous system or the parasympathetic nervous system are stimulated or not. For example, while the reflected wave time of the blood pulse signal decreases, the sympathetic nervous system is stimulated. On the other hand, while the reflected wave time of the blood pulse signal increases, the parasympathetic nervous system is stimulated.

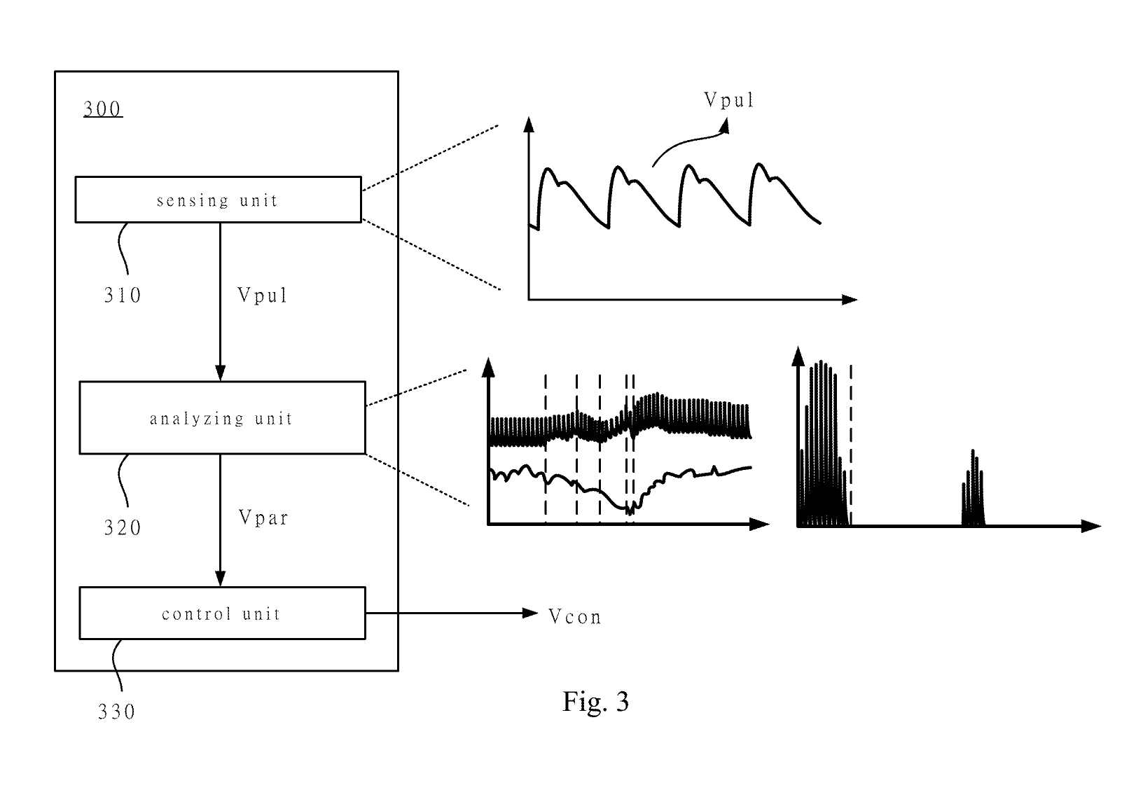

As shown in FIG. 2, the amplitude of the blood pulse signal decreases from the original magnitude A1 to magnitude A2 in period T12, which is after period T11, and the reflected wave time of the blood pulse signal also decreases in period T12. That is to say, the ejection fraction decreases and the sympathetic nervous system is stimulated in period T12. And then because of the ending of performing the valsalva maneuver, the blood pulse signal increases from the magnitude A2 to magnitude A3 in period T2, which is after period T12, and the reflected wave time of the blood pulse signal also increases in period T2. That is to say, the ejection fraction increases and the parasympathetic nervous system is stimulated in period T2. Therefore, a cardiac regulating apparatus 300 shown in FIG. 3 is configured to determine the information of the ejection fraction and the autonomic nervous systems by the acquired amplitude and the reflected wave time of the blood pulse signal.

FIG. 3 is a schematic diagram illustrating the cardiac regulating apparatus 300 according to an embodiment of the present disclosure. The cardiac regulating apparatus 300 includes a sensing unit 310, an analyzing unit 320 and a control unit 330. The control unit 330 is configured for outputting a cardiac control signal Vcon in accordance with a heart rate of a heart, that is to say, the cardiac regulating apparatus 300 is set up in a human body (of a patient) for stabilizing the heart rate in the embodiment. The cardiac control signal Vcon has a first constant cardiac rhythm such as 80 Hz, and after the cardiac regulating apparatus 300 is set up in the human body, the cardiac regulating apparatus 300 may stimulate the heart according to the first constant cardiac rhythm.

As shown in FIG. 3, the sensing unit 310 is configured for acquiring a blood pulse signal Vpul. The sensing unit 310 can be a photoelectric sensor such as a red-light transceiver or a infrared transceiver, a piezoelectric sensor or another equivalent sensor for acquiring the blood pulse signal Vpul. The sensing unit 310 can be an intrusive sensor or a non-intrusive sensor, and the present disclosure is not limited in this regard.

While the cardiac control signal Vcon having the first constant cardiac rhythm, the analyzing unit 320 is configured for analyzing amplitude and reflected wave time of the blood pulse signal Vpul as shown in FIG. 2, and then outputting a regulation parameter Vpar according to the amplitude and the reflected wave time of the blood pulse signal Vpul. The control unit 330 is configured for regulating the cardiac control signal Vcon according to the regulation parameter Vpar from the first constant cardiac rhythm to a second constant cardiac rhythm different from the first constant cardiac rhythm. For example, in period T12 shown in FIG. 2 and FIG. 3, while the analyzing unit 320 receives the information indicating that the amplitude of the blood pulse signal Vpul decreases from the original magnitude A1 to the magnitude A2 (i.e., the information indicating that the ejection fraction decreases), the analyzing unit 320 may output the regulation parameter Vpar. And content in the regulation parameter Vpar can be increasing 5 Hz or increasing 10 Hz of the first constant cardiac rhythm, and the present disclosure is not limited in this regard. Therefore, after the control unit 330 receives the regulation parameter Vpar, the original first constant cardiac rhythm (such as 80 Hz) of the cardiac control signal Vcon will be increased to the second cardiac rhythm (such as 85 Hz or 90 Hz). For another example, in period T2, while the analyzing unit 320 receives the information indicating that the amplitude of the blood pulse signal Vpul increases from the magnitude A2 to the magnitude A3 (i.e., the information indicating that the ejection fraction increases), the analyzing unit 320 may output the regulation parameter Vpar. And content in the regulation parameter Vpar can be decreasing 5 Hz or decreasing 10 Hz of the first constant cardiac rhythm, and the present disclosure is not limited in this regard. Therefore, after the control unit 330 receives the regulation parameter Vpar, the original first constant cardiac rhythm (such as 90 Hz) of the cardiac control signal Vcon will be decreased to the second cardiac rhythm (such as 85 Hz or 80 Hz). It should be noted that, the second constant cardiac rhythm in period T12 is the first constant cardiac rhythm in the next period T2, but the present disclosure is not limited in this regard, the second constant cardiac rhythm at any moment can be the first constant cardiac rhythm at the next moment.

In addition, in period T12, while the analyzing unit 320 receives the information indicating that the reflected wave time of the blood pulse signal Vpul decreases (i.e., the information indicating that the sympathetic nervous system is stimulated), the analyzing unit 320 may output the regulation parameter Vpar. And content in the regulation parameter Vpar can be increasing 5 Hz or increasing 10 Hz of the first constant cardiac rhythm, and the present disclosure is not limited in this regard. Therefore, after the control unit 330 receives the regulation parameter Vpar, the original first constant cardiac rhythm (such as 80 Hz) of the cardiac control signal Vcon will be increased to the second cardiac rhythm (such as 85 Hz or 90 Hz). For another example, in period T2, while the analyzing unit 320 receives the information indicating that the reflected wave time of the blood pulse signal Vpul increases (i.e., the information indicating that the parasympathetic nervous system is stimulated), the analyzing unit 320 may output the regulation parameter Vpar. And content in the regulation parameter Vpar can be decreasing 5 Hz or decreasing 10 Hz of the first constant cardiac rhythm, and the present disclosure is not limited in this regard. Therefore, after the control unit 330 receives the regulation parameter Vpar, the original first constant cardiac rhythm (such as 90 Hz) of the cardiac control signal Vcon will be decreased to the second cardiac rhythm (such as 85 Hz or 80 Hz). Similarly, the second constant cardiac rhythm in period T12 is the first constant cardiac rhythm in the next period T2, but the present disclosure is not limited in this regard, the second constant cardiac rhythm at any moment can be the first constant cardiac rhythm at the next moment. It should be noted that, the amplitude and the reflected wave time of the blood pulse signal Vpul can independently influence the regulation parameter Vpar, that is to say, the analyzing unit 320 can output the regulation parameter Vpar according to one of the two (the amplitude and the reflected wave time) or both of the two.

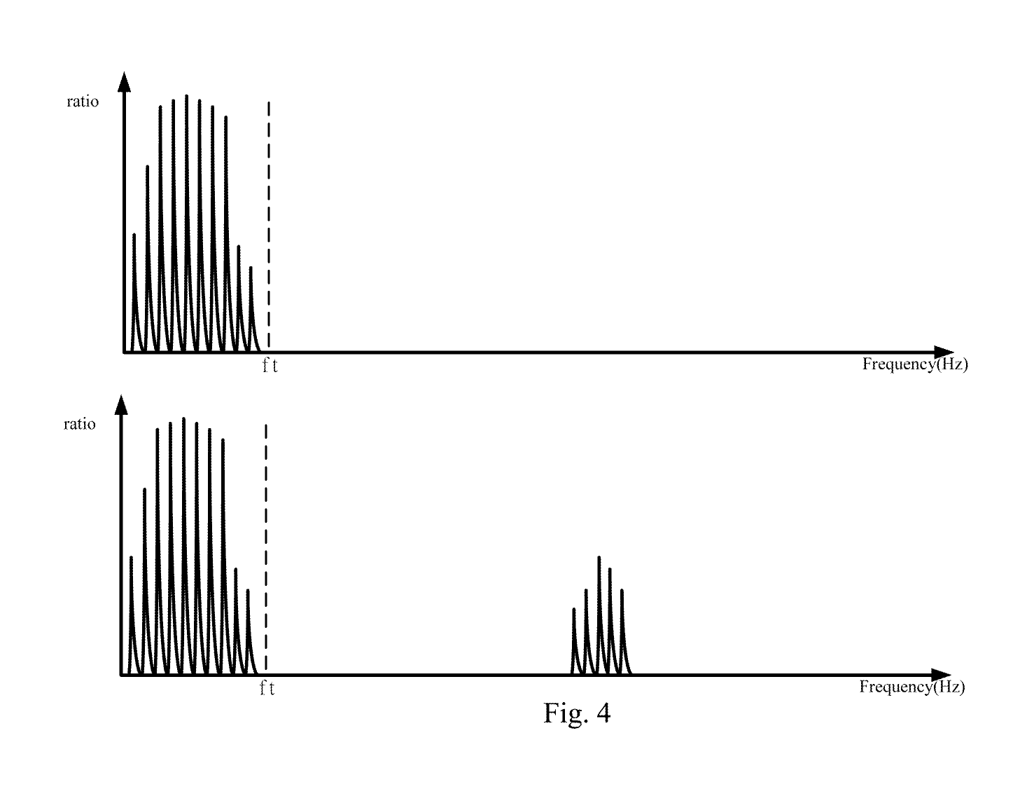

In some embodiments, the analyzing unit 320 further includes a spectrum analyzing module (which is not shown in FIG. 3) configured for analyzing spectrum values of the blood pulse signal Vpul. While variations of the blood pulse signal Vpul are small, the spectrum values may all be lower than a predetermined value ft as shown in the upper-portion of FIG. 4. However, while variations of the blood pulse signal Vpul are large, there will be a portion of the spectrum values falling higher than the predetermined value ft as shown in the lower-portion of FIG. 4. In practice, the variations of the blood pulse signal Vpul may cause variations of the regulation parameter Vpar, that is to say, while the variations of the blood pulse signal Vpul are large, the variations of the regulation parameter Vpar may also be large. Therefore, the spectrum values obtained by the analyzing unit 320 can be regarded as the variations of the regulation parameter Vpar.

In order to prevent variations of the heart rate in the human body from being too large during regulation, the control unit 330 may decrease variations of the cardiac control signal Vcon while a spectrum value of the blood pulse signal Vpul is higher than the predetermined value ft. For example, while the spectrum value of the blood pulse signal Vpul is higher than the predetermined value ft such as 0.3 Hz, the control unit 330 may determine that the variations of the regulation parameter Vpar are too large, in which the content in the regulation parameter Vpar may be increasing 20 Hz of the first constant cardiac rhythm. In other words, the content in the regulation parameter Vpar may be decreasing 20 Hz of the first constant cardiac rhythm at the last moment, and quickly changed to be increasing 20 Hz of the first constant cardiac rhythm at the moment. Therefore, the control unit 330 may decrease the variations of the cardiac control signal Vcon, for example, the control unit 330 restricts the variations down to 50% of the original regulation parameter Vpar. In this example, the content in the regulation parameter Vpar may be restricted from increasing 20 Hz to increasing 10 Hz of the first constant cardiac rhythm. Thus, the first constant cardiac rhythm (such as 80 Hz) of the cardiac control signal Vcon will be increased only 10 Hz instead of 20 Hz to the second constant cardiac rhythm (such as 90 Hz).

It should be noted that, while the spectrum values of the blood pulse signal Vpul are all lower than the predetermined value ft such as 0.3 Hz, the control unit 330 may recover the variations of the cardiac control signal, i.e., stopped restricting the variations of the cardiac control signal.

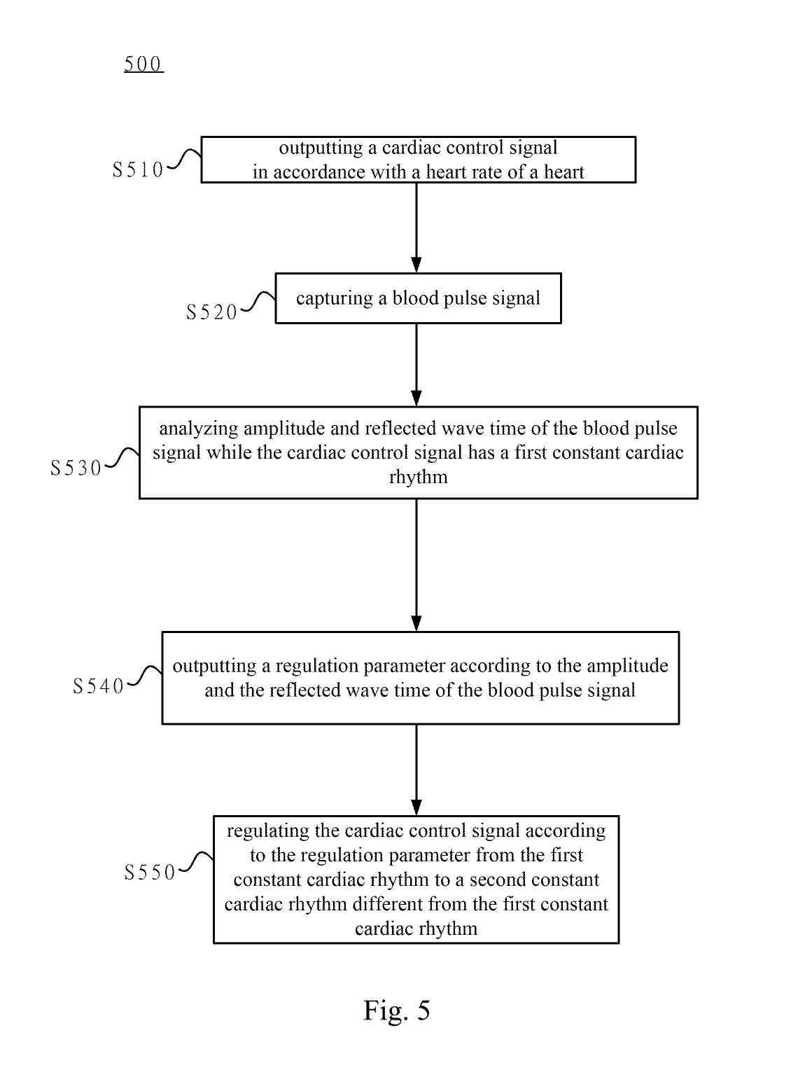

FIG. 5 is a schematic diagram illustrating a cardiac regulating method 500 according to an embodiment of the present disclosure. The cardiac regulating method 500 can be utilized in the cardiac regulating apparatus 300 in the abovementioned embodiment, but the present disclosure is not limited in this regard, the cardiac regulating method 500 can be utilized in another equivalent electronic apparatus.

As shown in FIG. 5, firstly, step S510 is executed for outputting a cardiac control signal in accordance with a heart rate of a heart.

Afterward, step S520 is executed for acquiring a blood pulse signal.

Afterward, step S530 is executed for analyzing amplitude and reflected wave time of the blood pulse signal while the cardiac control signal has a first constant cardiac rhythm.

Afterward, step S540 is executed for outputting a regulation parameter according to the amplitude and the reflected wave time of the blood pulse signal.

Afterward, step S550 is executed for regulating the cardiac control signal according to the regulation parameter from the first constant cardiac rhythm to a second constant cardiac rhythm different from the first constant cardiac rhythm.

To summarize, the present disclosure provides a cardiac regulating apparatus and a cardiac regulating method thereof. The cardiac control signal can be regulated for different physiological needs by analyzing the ejection fraction and the autonomic nervous systems in a non-intrusive manner through the magnitude and the reflected wave time of the blood pulse signal.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A cardiac regulating apparatus, comprising:
a control unit, configured for outputting a cardiac control signal in accordance with a heart rate of a heart;
a sensing unit, configured for acquiring a blood pulse signal; and
an analyzing unit, in response to the cardiac control signal having a first constant cardiac rhythm, the analyzing unit being configured for analyzing amplitude and reflected wave time of the blood pulse signal, and outputting a regulation parameter according to the amplitude and the reflected wave time of the blood pulse signal, wherein the control unit is configured for regulating the cardiac control signal according to the regulation parameter from the first constant cardiac rhythm to a second constant cardiac rhythm different from the first constant cardiac rhythm.

2. The cardiac regulating apparatus of claim 1, wherein the regulation parameter has a time-varying spectrum value, and the control unit is configured for regulating the cardiac control signal according to the regulation parameter and the spectrum value from the first constant cardiac rhythm to the second constant cardiac rhythm.

3. The cardiac regulating apparatus of claim 1, wherein, in response to the amplitude or the reflected wave time of the blood pulse signal being decreased, the analyzing unit increases the regulation parameter.

4. The cardiac regulating apparatus of claim 1, wherein, in response to the amplitude or the reflected wave time of the blood pulse signal being increased, the analyzing unit decreases the regulation parameter.

5. The cardiac regulating apparatus of claim 2, wherein, in response to the spectrum value being higher than a predetermined value, the control unit decreases variations of the cardiac control signal.

6. A cardiac regulating method, comprising:
outputting a cardiac control signal in accordance with a heart rate of a heart;
acquiring a blood pulse signal;
analyzing amplitude and reflected wave time of the blood pulse signal in response to the cardiac control signal having a first constant cardiac rhythm;
outputting a regulation parameter according to the amplitude and the reflected wave time of the blood pulse signal; and
regulating the cardiac control signal according to the regulation parameter from the first constant cardiac rhythm to a second constant cardiac rhythm different from the first constant cardiac rhythm.

7. The cardiac regulating method of claim 6, wherein the regulation parameter has a time-varying spectrum value, and the cardiac regulating method further comprises regulating the cardiac control signal according to the regulation parameter and the spectrum value from the first constant cardiac rhythm to the second constant cardiac rhythm.

8. The cardiac regulating method of claim 6, wherein, in response to the amplitude or the reflected wave time of the blood pulse signal being decreased, the cardiac regulating method further comprises increasing the regulation parameter.

9. The cardiac regulating method of claim 6, wherein,
in response to the amplitude or the reflected wave time of the blood pulse signal being increased, the cardiac regulating method further comprises decreasing the regulation parameter.

10. The cardiac regulating method of claim 7, wherein,
in response to the spectrum value being higher than a predetermined value, the cardiac regulating method further comprises decreasing variations of the cardiac control signal.
